(19) United States
(12) Patent Application Publication
(10) Pub. No.: US 2006/0051821 A1
(43) Pub. Date: Mar. 9, 2006

(54) Methods and kits for use in the diagnosis and treatment of endotoxemia

(76) Inventors: Daniel P Rossignol, Mahwah, NJ (US); Melvyn Lynn, East Brunswick, NJ (US)

Correspondence Address: CLARK & ELBING LLP, 101 FEDERAL STREET, BOSTON, MA, 02110, US

(21) Appl. No.: 10/545,136

(22) PCT Filed: Feb. 12, 2004
(86) PCT No.: PCT/US04/04552

§ 371 Date: Aug. 10, 2005

Related U.S. Application Data
(60) Provisional application No. 60/446,891, filed on Feb. 12, 2003.

(51) Int. Cl. G01N 33/554; G01N 33/569

(52) U.S. Cl. 435/7.32

(57) ABSTRACT

The invention provides methods and kits for use in determining whether a patient may benefit from treatment with a toll-like receptor 4 (TLR4) antagonist, such as an antiendotoxin compound.

### FIELD OF THE INVENTION

This invention relates to diagnostic methods and kits for use in determining whether to administer toll-like receptor 4 (TLR4) antagonists, such as antiendotoxin drugs, to patients.

### BACKGROUND OF THE INVENTION

The incidence of sepsis due to infection in the United States has been estimated to be approximately 750,000 cases per year, with a mean mortality rate of about 30% (Angus et al., Crit. Care Med. 29:1303-1310, 2001). Endotoxin, or lipopolysaccharide (LPS), which is a major component of the cell wall of gram-negative bacteria, is the causative agent of gram-negative sepsis. Endotoxin induces an innate immune response through toll-like receptor 4 (TLR4) (Medzhitov et al., Nature 388(6640):394-397, 1997) in infected hosts, by which the body is warned of the bacterial infection, thus leading to an antimicrobial attack by the host immune system. Such an immune response is usually beneficial to infected hosts, however, an overwhelming immune response to endotoxin can be pathological, leading to systemic inflammatory response syndrome (SIRS), organ failure, and, possibly, septic shock and death. The symptoms of these conditions include fever, generalized inflammation, and more severe conditions, such as disseminated intravascular coagulation (DIC), hypotension, acute renal failure, acute respiratory distress syndrome (ARDS), hepatocellular destruction, and cardiac failure. Given the prevalence and potential severity of gram-negative sepsis, the importance of rapid and accurate methods for diagnosing this condition, to enable prompt treatment, cannot be overstated.

While endotoxin itself is a highly heterogeneous molecule, the expression of many of the toxic properties of endotoxin is attributed to the highly conserved hydrophobic lipid A portion. An effective drug that acts as a TLR4 antagonist, and which is an antagonist to this conserved structure of Lipid A, is known as E5564 (also known as compound 1287, SGEA, and Eriforan)(Mullarkey et al., J. Pharmacol. Exp. Ther. 304(3):1093-1102, 2003). This drug is described as compound 1 in U.S. Pat. No. 5,681,824, which is incorporated herein by reference. E5564 has the formula: (α-D-Glucopyranose, 3-O-decyl-2-deoxy-6-O-[2-deoxy-3-O—[(3R)-3-methoxydecyl)-6-O-methyl-2-[[(11Z)-1-oxo-11-octadecenyl)amino]-4-O-phosphono-β-D-glucopyranosyl]-2-[(1,3-dioxotetradecyl)amino]-,1-(dihydrogen phosphate), which can be provided as a tetrasodium salt. E5564 has a molecular weight of 1401.6.

### SUMMARY OF THE INVENTION

The invention provides methods for determining whether a patient (e.g., an intensive care unit patient) could benefit or continue to benefit from treatment with a TLR4 antagonist, such as an antiendotoxin compound. The methods include the following steps: (a) contacting a sample from the patient that includes infected tissue or a fluid containing white blood cells (e.g., a whole blood sample) with an antibody (e.g., an IgM antibody) that specifically binds to an indicator of gram-negative bacterial infection (e.g., endotoxin); and (b) detecting the level of oxidants produced by white blood cells in the sample in the presence of the antibody as a measure of the level of the indicator in the sample. Detection of an increased level of the indicator in the sample, relative to a control, indicates that the patient could benefit from treatment with a TLR4 antagonist, such as an antiendotoxin compound. Preferably, these methods are conducted by use of a bedside kit. Also, preferably, the TLR4 antagonist that may be indicated by use of the methods of the invention has the following structure:

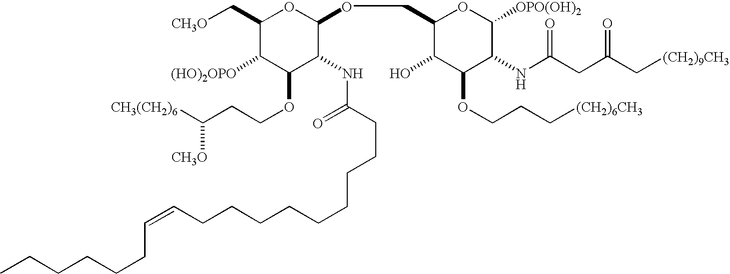

The invention also includes kits for use in determining whether patients could benefit from treatment with a TLR4 antagonist, such as an antiendotoxin compound. The kits may contain the following components: (a) a first container including an antibody (e.g., an IgM antibody) that specifically binds to an indicator of gram-negative bacterial infection (e.g., endotoxin); (b) a second container including a chemiluminescent compound; (c) a third container including zymosan or latex beads (which, preferably, are opsonized); and (d) instructions for using the kits to determine whether patients could benefit from treatment with TLR4 antagonists, such as antiendotoxin compounds. Optionally, the kits may also include a fourth container including the indicator.

The invention provides several advantages. For example, the methods of the invention facilitate rapid and convenient determination of whether it may be beneficial to administer a TLR4 antagonist, such as an antiendotoxin compound to a patient, so that the effects of complications related to endotoxemia can be treated or prevented in the patient. The rapidity of the methods enables treatment to begin promptly, if necessary, so that the health of a patient does not worsen while the patient awaits results from other tests that may take longer. The methods of the invention also facilitate monitoring of the efficacy of treatment with a TLR4 antagonist because, as is discussed further below, the presence of the drug in a patient sample does not necessarily interfere with analysis of endotoxin levels in the sample. In addition, the methods used in the invention are very sensitive, allowing the detection of infection or endotoxemia due to organ dysfunction or disease (e.g., liver failure, gut injury, or pancreatic injury) at an earlier time point than would be possible for methods requiring the detection of antibodies.

Other features and advantages of the invention will be apparent from the following detailed description and the claims.

### DETAILED DESCRIPTION

The invention provides diagnostic methods and kits for use in determining whether a patient could benefit from treatment with a TLR4 antagonist, such as an antiendotoxin compound, or if treatment with such a compound should be discontinued. The methods of the invention employ an assay that involves determination of the level of an indicator of the presence of endotoxin or another TLR4 agonist due to gram-negative bacterial infection or organ dysfunction in a patient sample. In particular, a patient sample that includes white blood cells (e.g., whole blood) is combined with an antibody that specifically recognizes the indicator, and complexes that form between the antibody and any indicator that is present in the sample trigger a response in the white blood cells of the sample, which can be used as a measure of the presence of the indicator in the sample. Detection of an increased amount of the indicator in a patient sample using this approach thus indicates that treatment with a TLR4 antagonist, such as an antiendotoxin compound, may be advisable. The invention also includes kits that can be used to carry out these methods. The methods and kits of the invention are described in further detail below.

As is noted above, the control and treatment of endotoxemia or sepsis due to gram-negative bacterial infection is a significant problem in medicine. This problem arises in many contexts, most notably with patients who are being treated in hospital intensive care units, patients having compromised immune systems, and surgical patients. Samples from these patients, as well as other patients that are at risk of endotoxemia or gram-negative bacterial infection, can be analyzed using the methods of the present invention, to determine whether treatment using a TLR4 antagonist, such as an antiendotoxin compound, may be advisable.

The methods of the invention can employ any of a number of different formats. Preferable formats are those that are amenable to bedside use, which enable medical professionals to determine rapidly, conveniently, and inexpensively whether a patient has higher than normal levels of an indicator of gram-negative bacterial infection with minimal, if any, use of a medical laboratory. Indeed, the methods used in the present invention are particularly appropriate for this type of format. However, formats that require the use of a medical laboratory are also included in the invention.

A preferred indicator of gram-negative bacterial infection that is detected using the methods of the invention is endotoxin, which, as is noted above, is the major lipopolysaccharide component of the cell wall of gram-negative bacteria and the causative agent of gram-negative sepsis. Other indicators that can be detected using the methods of the invention include those that are produced by a host in response to infection, such as, for example, TNF-α, IL-1, and IL-6, or endogenously derived TLR4 agonists such as those that arise from endotoxemia, for example.

Details of methods that can be used to determine the level of an indicator of gram-negative bacterial infection in a sample, for use in the methods of the present invention, are provided in, for example, U.S. Pat. No. 5,804,370, which is incorporated herein by reference (also see, for example, WO 01/04633 and Romaschin et al., J. Immunol. Methods 212:169-185, 1998). Briefly, a patient sample containing white blood cells and related complement proteins is obtained from a patient. Preferably, the sample is a whole blood sample, but it may also be any other type of sample containing these components as well. For example, a plasma, serum, or other sample that includes neutrophils, lymphocytes, and/or monocytes may also be used.

The sample, which preferably is anticoagulated with heparin or EDTA, in the case of whole blood, is mixed with an antibody that specifically recognizes the indicator of gram-negative bacterial infection. Any of a number of commercially available antibodies can be used for this purpose or, of course, those of skill in this art can produce their own indicator-specific antibodies, using standard methods. For example, in the case of endotoxin, the antibodies can be specific for the lipid A region of endotoxin (e.g., Xomen-E5 (Xoma, Inc., Palo Alto, Calif.), which is a murine monoclonal IgM pentamer directed against the lipid A portion of gram-negative endotoxin), or, alternatively, can be specific for another portion of the molecule, such as the polysaccharide portion. Use of antibodies with the latter type of specificity may be particularly useful in monitoring treatment with E5564 (see above), as the drug does not include a region that corresponds to the polysaccharide portion of endotoxin.

The antibody used in the invention can be of any type or isotype but, preferably, is a monoclonal antibody of the IgM or IgG isotype. In the case of IgM antibodies, for example, complexes formed between these antibodies and endotoxin result in the activation of the classical complement pathway. A heat labile element is produced in this pathway, which activates the production of oxidants by white blood cells in the patient sample. Detection of the level of oxidants produced is then used as a measure of the level of the indicator in the sample. The oxidants are detected by incubation of the sample/antibody mixture with a chemiluminescent compound, such as, for example, luminol, lucigenin, or pholasin. These compounds emit light at a level that is proportional to the amount of oxidants present in the sample, which is measured by use of a luminometer. Preferably, the method described above is carried out in the presence of zymosan and/or latex beads, which, preferably, are opsonized. The presence of these components in the reaction enhances the chemiluminescent response by stimulating oxidant production by white blood cells, as well as phagocytosis. In addition, the order in which the components of a reaction mixture are added can vary, as can be determined by those of skill in this art. For example, the chemiluminescent substrate can be added to the blood sample prior to addition of the antibody.

Appropriate controls for use in the methods of the invention can readily be selected by those of skill in this art. For example, a non-specific antibody of the same isotype as that which is specific for the indicator molecule can be used with a sample that is processed in parallel with a test sample as a negative control. As another example, a positive control including exogenously added indicator can be used. Detection of a statistically significant increase in oxidant production in a test sample, as compared to a negative control, indicates the presence of the indicator in the sample, thus providing a medical professional with a basis for determining whether a TLR4 antagonist, such as an antiendotoxin compound should be administered.

The invention also includes kits that can be used to conduct any of the methods that are described above. Such kits thus include, for example, a first container of antibody (e.g., IgM antibody) that is specific for an indicator of sepsis (e.g., endotoxin), a second container of a chemiluminescent compound (e.g., luminol), and, optionally, a third container of zymoxan or latex beads and/or a fourth container of control indicator. The kits also include instructions for a medical professional to follow in order to determine a level of indicator that would indicate treatment with a TLR4 antagonist, such as an antiendotoxin compound.

Antiendotoxin compounds that can be used to treat a patient identified as in need of such treatment, according to the invention, include, for example, Compound E5564 (also known as compound 1287, SGEA, and Eriforan; U.S. Pat. No. 5,935,938) and Compound B531 (U.S. Pat. No. 5,530,113), as well as other compounds described in these patents and the following U.S. patents: U.S. Pat. No. 5,612,476, U.S. Pat. No. 5,756,718, U.S. Pat. No. 5,843,918, U.S. Pat. No. 5,750,664, and U.S. Pat. No. 5,681,824. For example, the antiendotoxin compound can have the formula: 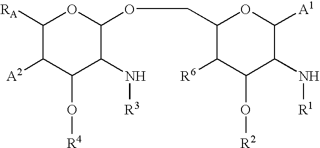
where R¹ is selected from the group consisting of: 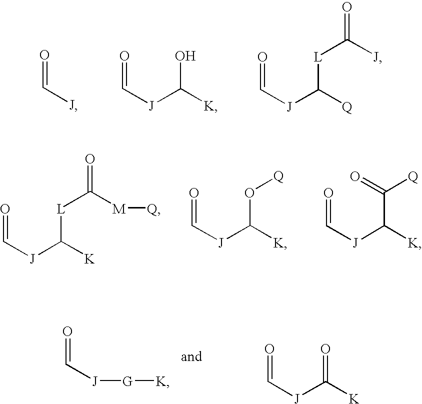
where each J, K, and Q, independently, is straight or branched C1 to C15 alkyl; L is O, NH, or CH₂; M is O or NH; and G is NH, O, S, SO, or SO₂,
R² is straight or branched C5 to C15 alkyl;
R³ is selected from the group consisting of straight or branched C5 to C18 alkyl, 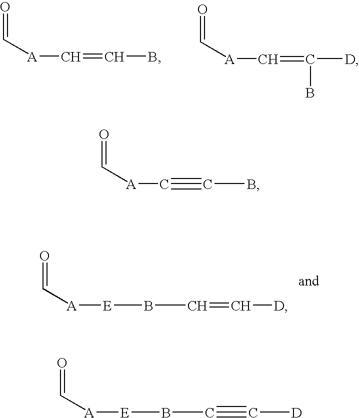
   where E is NH, O, S, SO, or SO₂; each A, B, and D, independently, is straight or branched C1 to C15 alkyl;
R⁴ is selected from the group consisting of straight or branched C4 to C20 alkyl, and 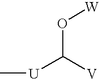
   where each U and V, independently, is straight or branched C2 to C15 alkyl and W is hydrogen or straight or branched C1 to C5 alkyl;
R_{A} is R⁵ or R⁵—O—CH₂—, R⁵ being selected from the group consisting of hydrogen, J′,-J′-OH, -J′-O—K′,-J′-O—K′—OH, and -J′-O—PO(OH)₂, where each J′ and K′, independently, is straight or branched C1 to C5 alkyl;
R⁶ is selected from the group consisting of hydroxy, halogen, C1 to C5 alkoxy, and C1 to C5 acyloxy;
A¹ and A², independently, are selected from the group consisting of 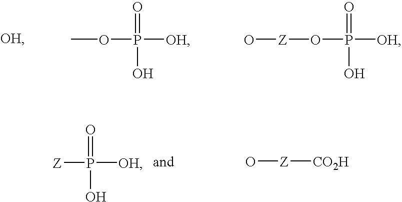
   where Z is straight or branched C1 to C10 allyl;
or pharmaceutically acceptable salts thereof.

A preferred, specific example of a compound that can be used in the invention, compound E5564 (also known as compound 1287, SGEA, and Eriforan), has the following structure:

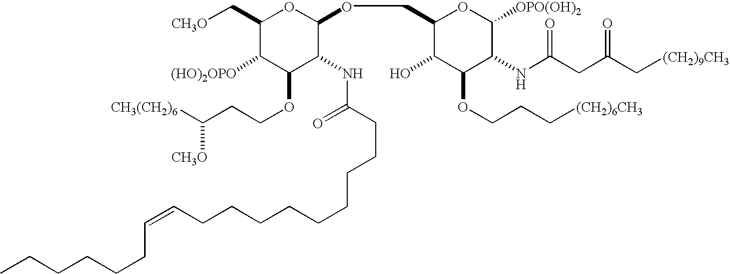

As is noted above, the methods of the invention can be used with patients undergoing any type of surgery or medical procedure that could lead to the occurrence of endotoxemia or sepsis syndrome, for example, cardiac surgery (e.g., cardiac bypass, cardiopulmonary bypass, or valve replacement), abdominal surgery, transplantation (of, e.g., liver, heart, kidney, or bone marrow), cancer surgery (e.g., removal of a tumor), major orthopedic surgery (e.g., revision hip arthroplasty or fusion/instrumentation of multiple lumbar or thoracic vertebrae), major general surgery (e.g., any laparotomy expected to exceed two hours duration, including partial hepatectomy, pancreatic surgery, or colon surgery), major urological surgery (e.g., radical cystectomy or radical nephrectomy), major vascular surgery (e.g., abdominal aortic aneurysm repair), and major gynecological surgery (e.g., cancer debulking procedure or abdominal hysterectomy with oophorectomy). Additional examples of surgical procedures with which the methods of the invention can be used are surgery for treating acute pancreatitus or inflammatory bowel disease, placement of a transjugular intrahepatic portosystemic stent shunt, hepatic resection, burn wound revision, and burn wound escharectomy.

The methods of the invention can also be used in conjunction with non-surgical procedures in which the gastrointestinal tract is compromised. For example, the methods of the invention can be used in association with chemotherapy or radiation therapy in the treatment of cancer, or with patients that have undergone any type of physical trauma.

The methods of the invention can also be used to determine whether it may be advisable to administer a TLR4 antagonist, such as an antiendotoxin compound, to a subject who has pulmonary bacterial infection or symptomatic pulmonary exposure to endotoxin and related disorders and conditions. Examples of such disorders and conditions include, for example, cystic fibrosis; immune deficiencies, including immunocompromise due to anti-cancer therapy and immunocompromise due to anti-rejection therapy after organ transplant; asplenia; hypogammaglobulinemia; dysglobulinemias; deficiencies of complement cascade components; HIV or other viral infections; polymorphonuclear granulocyte defects; ciliary dyskinesias (e.g., Kartagener's syndrome); obstructive pulmonary disorders, including congestive heart failure with pulmonary edema, chronic obstructive pulmonary disease, tumors leading to bronchial obstruction, and bronchiectasis (e.g., as a complication of asthma); acute lung injuries that predispose to infection, increase sensitivity to endotoxin, or affect ability to clear endotoxin (e.g., smoke inhalation or heat exposure (e.g., thermal injury, such as by inhalation of hot air or steam); aspiration of gastric contents; near-drowning; inhalation of noxious substances; and surgical or other medical treatments that result in the release of endotoxin or other TLR4 agonists.

TLR4 antagonists, such as antiendotoxin compounds, can be administered to patients identified as being in need of such treatment, according to the methods of the invention, using routes (e.g., injection, infusion, or inhalation) and dosages that are determined to be appropriate by those of skill in this art. For example, in the case of surgical patients, the drug can be administered intravenously for 1-6 hours preoperatively, and administration can be continued for up to 72 hours (e.g., 24 hours) postoperatively. The dose can be, for example, 2-10,000 μg/hour, e.g., 25-3,000 μg/hour, or, preferably, 50-1,000 μg/hour. Alternatively, the drug can be administered only preoperatively, operatively, postoperatively, or any combination thereof. The drug is administered in a pharmaceutically acceptable formulation, which may include 5% glucose.

In the case of a patient that has pulmonary bacterial infection, symptomatic pulmonary exposure to endotoxin, or a related disorder, the administration can be by inhalation by, e.g., the use of aerosol or a nebulizer. Such administration can be effected by means of periodic bolus administration, by continuous, metered inhalation, or by a combination of the two. A single dose is administered by inhalation of 1 μg-24 mg, for example, 5-150 μg, or, preferably, 10-100 μg of the drug. Of course, recalcitrant disease may require administration of relatively high doses, e.g., 5 mg, the appropriate amounts of which can be determined by those of skill in this art. Appropriate frequency of administration can also be determined by those of skill in this art, and can be, for example, 1-4, for example, 2-3, times each day. Preferably, the drug is administered once each day. In general, treatment is carried out until the level of the indicator decreases to an acceptable level and/or until any symptoms in the patient have lessened to a satisfactory extent (or, preferably, have disappeared), and it may be necessary, in some cases, to continue administration for several days, e.g., one, two, three, or four weeks.

As will be understood by those of skill in this art, the specific dose level for any particular patient will depend on a variety of factors, including the activity of the specific compound employed; the age, body weight, general health, and sex of the individual being treated; the time and route of administration; the rate of excretion; other drugs that have previously been administered; and the severity of the particular disease undergoing therapy.

All publications cited above are hereby incorporated by reference.

What is claimed is:

1. A method of determining whether a patient could benefit or continue to benefit from treatment with a toll-like receptor 4 (TLR4) antagonist, said method comprising the steps of:
(a) contacting a sample from said patient that comprises infected tissue or a fluid comprising white blood cells with an antibody that specifically binds to an indicator of gram-negative bacterial infection; and
(b) detecting the level of oxidants produced by white blood cells in said sample in the presence of said antibody as a measure of the level of said indicator in said sample, wherein detection of an increased level of said indicator in said sample, relative to a negative control, indicates that said patient could benefit from treatment with a TLR4 antagonist.

2. The method of claim 1, wherein the indicator is endotoxin.

3. The method of claim 1, wherein the patient sample is a whole blood sample.

4. The method of claim 1, wherein the antibody is an IgM antibody.

5. The method of claim 1, wherein the patient is an intensive care unit patient.

6. The method of claim 1, wherein the immunoassay is conducted by use of a bedside kit.

7. The method of claim 1, wherein the TLR4 antagonist is an antiendotoxin compound.

8. The method of claim 7, wherein the antiendotoxin compound comprises the following structure:

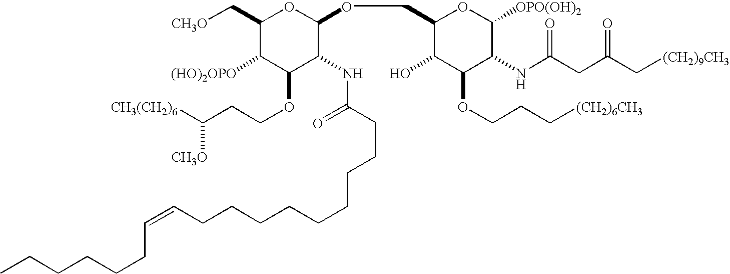

9. A kit for determining whether a patient could benefit from treatment with a TLR4 antagonist, said kit comprising:
(a) a first container comprising an antibody that specifically binds to an indicator of gram-negative bacterial infection;
(b) a second container comprising a chemiluminescent compound; and
(c) instructions for using said kit to determine whether a patient could benefit from treatment with a TLR4 antagonist.

10. The kit of claim 9, wherein said indicator is endotoxin.

11. The kit of claim 9, wherein said antibody is an IgM antibody.

12. The kit of claim 9, further comprising a third container comprising zymosan or latex beads

13. The kit of claim 9, further comprising a fourth container comprising said indicator.

14. The kit of claim 9, wherein said TLR4 antagonist is an antiendotoxin compound.
